(19) United States
(12) Patent Application Publication
(10) Pub. No.: US 2005/0119221 A1
(43) Pub. Date: Jun. 2, 2005

(54) Use of a cationic polysaccharide to enhance biocidal efficacies

(76) Inventors: Erning Xia, Penfield, NY (US); Alyce K. Dobie, Williamson, NY (US); Joseph C. Salamone, Fairport, NY (US); Irene Quenville, Oswego, NY (US); Roya N. Borazjani, Fairport, NY (US); Zhenze Hu, Pittsford, NY (US)

Correspondence Address: RITA D. VACCA;BAUSCH & LOMB INCORPORATED, ONE BAUSCH & LOMB PLACE, ROCHESTER, NY, 14604-2701, US

(21) Appl. No.: 10/724,680

(22) Filed: Dec. 1, 2003

(51) Int. Cl. A61K 31/715; A61K 31/155

(52) U.S. Cl. 514/54; 514/635

(57) ABSTRACT

The use of a composition of one or more cationic polysaccharides in combination with one or more antimicrobial agents to disinfect contact lenses and preserve ophthalmic lens compositions is described. Ophthalmic lens solutions containing compositions of one or more cationic polysaccharides in combination with one or more antimicrobial agents and methods of making and using the same are also described.

### FIELD OF THE INVENTION

The present invention is directed toward the use of one or more cationic polysaccharides to enhance disinfection and preservation. More particularly, the present invention is directed toward the use of one or more cationic polysaccharides in combination with one or more antimicrobial agents to enhance disinfection and preservation of ophthalmic solutions and devices.

### BACKGROUND OF THE INVENTION

Contact lenses in wide use today fall into two general categories, hard and soft. The hard or rigid corneal type lenses are formed from materials prepared by the polymerization of acrylic esters, such as poly(methyl methacrylate) (PMMA). The gel, hydrogel or soft type lenses are made by polymerizing such monomers as 2-hydroxyethyl methacrylate (HEMA) or, in the case of extended wear lenses, by polymerizing silicon-containing monomers or macromonomers. Both the hard and soft types of contact lenses are exposed to a broad spectrum of microbes during normal wear and become soiled relatively quickly. Contact lenses whether hard or soft therefore require routine cleaning and disinfecting. Failure to routinely clean and disinfect contact lenses properly can lead to a variety of problems ranging from mere discomfort when being worn to serious ocular infections. Ocular infections caused by virulent microbes such as Pseudomonas aeruginosa can lead to loss of the infected eye(s) if left untreated or if allowed to reach an advanced stage before initiating treatment.

U.S. Pat. No. 4,758,595 discloses a contact lens disinfectant and preservative containing a biguanide or a water-soluble salt thereof in combination with a buffer, preferably a borate buffer, e.g., boric acid, sodium borate, potassium tetraborate, potassium metaborate or mixtures of the same.

U.S. Pat. No. 4,361,548 discloses a contact lens disinfectant and preservative containing dilute aqueous solutions of a polymer; namely, dimethyldiallylammonium chloride (DMDAAC) having molecular weights ranging from about 10,000 to 1,000,000. Amounts of DMDAAC homopolymer as low as 0.00001 percent by weight may be employed when an enhancer, such as thimerosal, sorbic acid or phenylmercuric salt is used therewith. Although lens binding and concomitant eye tissue irritation with DMDAAC were reduced, it was found in some users to be above desirable clinical levels.

Despite the availability of various commercially available contact lens disinfecting systems such as heat, hydrogen peroxide, biguanides, polymeric biguanides, quaternary ammonium polyesters, amidoamines and other chemical agents, there continues to be a need for improved disinfecting systems. Such improved disinfecting systems include systems that are simple to use, are effective against a broad spectrum of microbes, are non-toxic and do not cause ocular irritation as the result of binding to the contact lens material. There is a particular need in the field of contact lens disinfection and ophthalmic composition preservation for safe and effective chemical agents with antimicrobial activity.

### SUMMARY OF THE INVENTION

The present invention relates to enhanced biocidal activity of multi-purpose contact lens solutions, useful for cleaning, disinfecting, soaking, rinsing, wetting and conditioning all types of contact lenses, including rigid permeable contact lenses. More particularly, the present invention relates to the use of one or more cationic polysaccharides, which allows for the use of a lowered level, or a reduced amount, of one or more antimicrobial agents with enhanced disinfection and preservation of ophthalmic solutions and devices. It has been found that compositions including one or more cationic polysaccharides in combination with a lowered level of one or more antimicrobial agents exhibit excellent disinfecting and/or preservative effect, while also increasing lens wearer comfort. The polysaccharide-containing compositions of the present invention are also useful for preservation of ophthalmic compositions such as pharmaceuticals, artificial tears and comfort drops against microbial contamination.

The subject polysaccharide-containing compositions are effective in the manufacture of multi-purpose solutions that are non-toxic, simple to use and do not cause ocular irritation.

Accordingly, it is an object of the present invention to provide compositions with enhanced biocidal activity useful in the manufacture of ophthalmic disinfecting systems.

Another object of the present invention is to provide a method for using compositions with enhanced biocidal activity in the disinfection of medical devices.

Another object of the present invention is to provide compositions with enhanced biocidal activity useful in ophthalmic systems for disinfecting contact lenses.

Another object of the present invention is to provide compositions with enhanced biocidal activity useful in preserving ophthalmic systems from microbial contamination.

Another object of the present invention is to provide compositions with enhanced biocidal activity useful in ophthalmic systems for disinfecting contact lenses with reduced or eliminated eye irritation.

Another object of the present invention is to provide a method of making compositions with enhanced biocidal activity useful in ophthalmic systems.

Still another object of the present invention is to provide a method of making compositions with enhanced biocidal activity useful as disinfecting and preservative agents.

These and other objectives and advantages of the present invention, some of which are specifically described and others that are not, will become apparent from the detailed description and claims that follow.

### DETAILED DESCRIPTION OF THE INVENTION

The compositions of the present invention can be used with all contact lenses such as conventional hard and soft lenses, as well as rigid and soft gas permeable lenses. Such suitable lenses include both hydrogel and non-hydrogel lenses, as well as silicone and fluorine-containing lenses. The term “soft contact lens” as used herein generally refers to those contact lenses that readily flex under small amounts of force. Typically, soft contact lenses are formulated from polymers having a certain proportion of repeat units derived from monomers such as 2-hydroxyethyl methacrylate and/or other hydrophilic monomers, typically crosslinked with a crosslinking agent. However, newer soft lenses, especially for extended wear, are being made from high-Dk silicone-containing materials.

Compositions of the present invention comprise one or more cationic polysaccharides in combination with one or more antimicrobial agents. It is surprising that the subject polysaccharide-containing compositions exhibit excellent disinfecting and/or preservative effect even when employed with lower than standard levels or amounts of one or more antimicrobial agents. Standard total amounts of antimicrobial agents in lens care solutions are in the range of 0.5 parts per million (ppm) to 15 ppm. From five up to a thirty percent reduction of the standard total amount of antimicrobial agent may be used in compositions of the present invention to achieve a disinfecting amount.

The polysaccharide-containing compositions of the present invention are useful for disinfecting medical devices. For example, the subject polysaccharide-containing compositions are useful in contact lens care solutions for disinfecting contact lenses. Compositions of the present invention are preferably in solution in sufficient concentration to destroy harmful microorganisms on the surface of a contact lens within the recommended minimum soaking time. The recommended minimum soaking time is included in the package instructions for use of the solution. The term “disinfecting solution” does not exclude the possibility that the solution may also be useful as a preserving solution, or that the disinfecting solution may be useful for other purposes such as daily cleaning, rinsing, and storage of contact lenses, depending on the particular formulation containing the subject compositions. Additionally, compositions of the present invention can be used in conjunction with other known disinfecting or preserving compounds if desired.

Compositions of the present invention in solution are physiologically compatible or “ophthalmically safe” for use with contact lenses. Ophthalmically safe as used herein means that a contact lens treated with or in the subject solution is generally suitable and safe for direct placement on the eye without rinsing. The subject solutions are safe and comfortable for daily contact with the eye via a contact lens that has been wetted with the solution. An ophthalmically safe solution has a tonicity and pH that is compatible with the eye and comprises materials, and amounts thereof, that are non-cytotoxic according to ISO (International Standards Organization) standards and U.S. FDA (Food and Drug Administration) regulations. The solution should be sterile in that the absence of microbial contaminants in the product prior to release should be statistically demonstrated to the degree necessary for such products.

Compositions of the present invention include one or more cationic polysaccharides in combination with one or more antimicrobial agents. One or more cationic polysaccharides are present in the subject compositions in a total amount of from approximately 0.001 to approximately 0.5 percent by weight based on the total weight of the composition, but more preferably from about 0.005 to about 0.05 percent by weight. Suitable cationic polysaccharides for use in compositions of the present invention include for example but are not limited to variations of polyquaternium-10 such as for example Polymer JR 125™ (Dow Chemical Company, Midland, Mich.) having a 2 percent solution viscosity of 75-125 cPs and 1.5 to 2.2 percent nitrogen, Polymer JR 400™ (Dow Chemical Company) having a 2 percent solution viscosity of 300 to 500 cPs and 1.5 to 2.2 percent nitrogen, Polymer JR 30M™ (Dow Chemical Company) having a 1 percent solution viscosity of 1,000 to 2,500 cPs and 1.5 to 2.2 percent nitrogen, Polymer LR 400™ (Dow Chemical Company) having a 2 percent solution viscosity of 300 to 500 cPs and 0.8 to 1.1 percent nitrogen, Polymer LR 30M™ (Dow Chemical Company) having a 1 percent solution viscosity of 1,250 to 2,250 cPs and 0.8 to 1.1 percent nitrogen, and Polymer LK™ (Dow Chemical Company) having a 2 percent solution viscosity of 300 to 500 cPs and 0.8 to 1.1 percent nitrogen. The preferred cationic polysaccharide for use in the present invention is Polymer JR 125™ or Polymer JR 400™.

Compositions of the present invention likewise include one or more antimicrobial agents. One or more antimicrobial agents are present in the subject compositions in a total amount of from approximately 0.00005 to approximately 0.0015 percent by weight based on the total weight of the composition, but more preferably from about 0.0007 to about 0.0015 percent by weight. Suitable antimicrobial agents for use in the present invention include for example but are not limited to 1,1′-hexamethylene-bis[5-(p-chlorophenyl)biguanide] (Chlorhexidine) or water soluble salts thereof, 1,1′-hexamethylene-bis[5-(2-ethylhexyl)biguanide] (Alexidine) or water soluble salts thereof, poly(hexamethylene biguanide) (PHMB) or water soluble salts thereof, polyquaternium-1 and quaternary ammonium esters. Biguanides are described in U.S. Pat. Nos. 5,990,174; 4,758,595 and 3,428,576, each incorporated herein in its entirety by reference. The preferred antimicrobial agents due to their ready commercial availability are poly(aminopropyl biguanide) (PAPB), also commonly referred to as poly(hexamethylene biguanide) (PHMB), and 1,1′-hexamethylene-bis[5-(2-ethylhexyl)biguanide] (Alexidine).

Compositions of the present invention may optionally include one or more aminoalcohol buffers, such as ethanolamine buffers, present in a total amount of from approximately 0.02 to approximately 3.0 percent by weight based on the total weight of the composition. Suitable aminoalcohol buffers include for example but are not limited to monoethanolamine (MEA), diethanolamine (DEA), triethanolamine (TEA), 2-amino-2-methyl-1,3-propanediol (AMPD), 2-dimethylamino-2-methyl-1-propanediol (DMAM P), 2-amino-2-ethylpropanol (AEP), 2-amino-1-butanol (AB) and 2-amino-2-methyl-1-propanol (AMP), but preferably MEA, DEA or TEA.

Compositions of the present invention likewise include at least one surfactant that has known advantages in terms of cleaning efficacy and comfort. Surfactants are present in the subject compositions in a total amount of from approximately 0.001 to approximately 25.0 percent by weight based on the total weight of the composition, but more preferably from about 0.001 to about 5.0 percent by weight. Suitable surfactants include for example but are not limited to polyethers based upon poly(ethylene oxide)-poly(propylene oxide)-poly(ethylene oxide), i.e., (PEO-PPO-PEO), or poly(propylene oxide)-poly(ethylene oxide)-poly(propylene oxide), i.e., (PPO-PEO-PPO), or a combination thereof. PEO-PPO-PEO and PPO-PEO-PPO are commercially available under the trade names Pluronics™, R-Pluronics™, Tetronics™ and R-Tetronics™ (BASF Wyandotte Corp., Wyandotte, Mich.) and are further described in U.S. Pat. No. 4,820,352 incorporated herein in its entirety by reference. Suitable surfactants for use in the present composition should be soluble in the lens care solution, not become turbid, and should be non-irritating to eye tissues.

Another useful class of surfactants are the hydroxyalkylphosphonates (HAP), such as those disclosed in U.S. Pat. No. 5,858,937 (Richards et al.), available under the trade name Dequest® (Montsanto Co., St. Louis, Mo.), and most preferably Dequest® 2016.

Optionally, it may be desirable to include one or more water-soluble viscosity agents in the subject composition. Because of the demulcent effect of viscosity agents, the same have a tendency to enhance the lens wearer's comfort by means of a film on the lens surface cushioning impact against the eye. Suitable viscosity agents include for example but are not limited to cellulose polymers like hydroxyethyl or hydroxypropyl cellulose, carboxymethyl cellulose, povidone, polyvinyl alcohol and the like. Viscosity agents may be employed in amounts ranging from about 0.01 to about 4.0 weight percent or less.

Compositions of the present invention when in solution likewise include one or more buffers, or a buffering system in addition to the aminoalcohol buffer, to adjust the final pH of the solution. Suitable buffers include for example but are not limited to phosphate buffers, borate buffers, tris(hydroxymethyl)aminomethane (Tris) buffers, bis(2-hydroxyethyl) imino-tris(hydroxymethyl)methane (bis-Tris) buffers, sodium bicarbonate, and combinations thereof. A suitable buffering system for example may include at least one phosphate buffer and at least one borate buffer, which buffering system has a buffering capacity of 0.01 to 0.5 mM, preferably 0.03 to 0.45, of 0.01 N of HCl and 0.01 to 0.3, preferably 0.025 to 0.25, of 0.01 N of NaOH to change the pH one unit. Buffering capacity is measured by a solution of the buffers only. The pH of lens care solutions of the present invention is preferably maintained within the range of 5.0 to 8.0, more preferably about 6.0 to 8.0, most preferably about 6.5 to 7.8.

Compositions of the present invention likewise include one or more tonicity agents to approximate the osmotic pressure of normal lachrymal fluids, which is equivalent to a 0.9 percent solution of sodium chloride or 2.5 percent glycerin solution. Examples of suitable tonicity agents include but are not limited to sodium and potassium chloride, dextrose, mannose, glycerin, calcium and magnesium chloride. These agents are typically used individually in amounts ranging from about 0.01 to 2.5 percent w/v and, preferably, from about 0.2 to about 1.5 percent w/v. Preferably, the tonicity agent is employed in an amount to provide a final osmotic value of 200 to 450 mOsm/kg and more preferably between about 220 to about 350 mOsm/kg, and most preferably between about 220 to about 320 mOsm/kg.

Compositions of the present invention may optionally include one or more sequestering agents to bind metal ions, which in the case of ophthalmic solutions, might otherwise react with protein deposits and collect on contact lenses. Suitable sequestering agents include for example but are not limited to ethylenediaminetetraacetic acid (EDTA) and its salts. Sequestering agents are preferably used in amounts ranging from about 0.01 to about 0.2 weight percent.

The compositions of the present invention are described in still greater detail in the examples that follow.

### EXAMPLE 1

### Preparation of Test Sample Solutions

Sample solutions for testing were prepared in accordance with the formulations set forth below in Table 1.

TABLE 1
Test Sample Solutions

| Ingredients | Sample | | | | |
|---|---|---|---|---|---|
| W/W Percent | 1 | 2 | 3 | 4 | 5 |
| Boric Acid | 0.8500 | 0.8500 | 0.8500 | 0.8500 | 0.8500 |
| Sodium Chloride | 0.1917 | 0.1917 | 0.1917 | 0.1917 | 0.1917 |
| Sodium Phosphate | 0.1500 | 0.1500 | 0.1500 | 0.1500 | 0.1500 |
| (monobasic) | | | | | |
| Sodium Phosphate | 0.3100 | 0.3100 | 0.3100 | 0.3100 | 0.3100 |
| (dibasic) | | | | | |
| Tetronic 1107 | 1.0000 | 1.0000 | 1.0000 | 1.0000 | 1.0000 |
| Pluronic F127 | 2.0000 | 2.0000 | 2.0000 | 2.0000 | 2.0000 |
| Polymer JR | 0.0200 | 0.0200 | 0.0200 | 0.0200 | 0.0200 |
| HAP (30%) | 0.1000 | 0.1000 | 0.1000 | 0.1000 | 0.1000 |
| Alexidine 2HCl | 2 ppm | 3 ppm | 4 ppm | 5 ppm | 6 ppm |
| Ingredients | Sample | | | | |
| W/W Percent | 6 | 7 | 8 | 9 | 10 |
| Boric Acid | 0.8500 | 0.8500 | 0.8500 | 0.8500 | 0.8500 |
| Sodium Chloride | 0.1917 | 0.1917 | 0.1917 | 0.1917 | 0.1917 |
| Sodium Phosphate | 0.1500 | 0.1500 | 0.1500 | 0.1500 | 0.1500 |
| (monobasic) | | | | | |
| Sodium Phosphate | 0.3100 | 0.3100 | 0.3100 | 0.3100 | 0.3100 |
| (dibasic) | | | | | |
| Tetronic 1107 | 1.0000 | 1.0000 | 1.0000 | 1.0000 | 1.0000 |
| Pluronic F127 | 2.0000 | 2.0000 | 2.0000 | 2.0000 | 2.0000 |
| Polymer JR | 0 | 0 | 0 | 0 | 0 |
| HAP (30%) | 0.1000 | 0.1000 | 0.1000 | 0.1000 | 0.1000 |
| Alexidine 2HCl | 2 ppm | 2.5 ppm | 3 ppm | 3.5 ppm | 4 ppm |

| Ingredients | Sample | | |
|---|---|---|---|
| W/W Percent | 11 | 12 | 13 |
| Boric Acid | 0.8500 | 0.8500 | 0.8500 |
| Sodium Chloride | 0.1917 | 0.1917 | 0.1917 |
| Sodium Phosphate | 0.1500 | 0.1500 | 0.1500 |
| (monobasic) | | | |
| Sodium Phosphate | 0.3100 | 0.3100 | 0.3100 |
| (dibasic) | | | |
| Tetronic 1107 | 1.0000 | 1.0000 | 1.0000 |
| Pluronic F127 | 2.0000 | 2.0000 | 2.0000 |
| Polymer JR | 0.0200 | 0.0200 | 0.0200 |
| HAP (30%) | 0.1000 | 0.1000 | 0.1000 |
| Alexidine 2HCl | 2 ppm | 2.5 ppm | 3 ppm |

| | |
|---|---|
| Ingredients | Sample |

| W/W Percent | 14 | 15 | 16 | 17 |
|---|---|---|---|---|
| Boric Acid | 0.8500 | 0.8500 | 0.8500 | 0.8500 |
| Sodium Chloride | 0.1917 | 0.1917 | 0.1917 | 0.1917 |
| Sodium Phosphate | 0.1500 | 0.1500 | 0.1500 | 0.1500 |
| (monobasic) | | | | |
| Sodium Phosphate | 0.3100 | 0.3100 | 0.3100 | 0.3100 |
| (dibasic) | | | | |
| Tetronic 1107 | 1.0000 | 1.0000 | 1.0000 | 1.0000 |
| Pluronic F127 | 2.0000 | 2.0000 | 2.0000 | 2.0000 |
| Polymer JR | 0 | 0.0200 | 0 | 0.0200 |
| HAP (30%) | 0.1000 | 0.1000 | 0.1000 | 0.1000 |
| Alexidine 2HCl | 4 ppm | 4 ppm | 5 ppm | 5 ppm |

### EXAMPLE 2

### Biocidal Testing of Test Samples With Five of FDA/ISO Challenge Microorganisms

Test solutions prepared in accordance with Example 1 above, were each tested for ISO/FDA microbial biocidal efficacy with 10 percent organic soil using five FDA/ISO challenge microorganisms, i.e., three bacteria and two fungi. Primary acceptance criteria established for bacteria require that the number of viable bacteria, recovered per ml, shall be reduced by a value not less than 3.0 logs within the minimum recommended disinfection period. Primary acceptance criteria established for yeasts and molds require that the number of viable yeasts and molds, recovered per ml, shall be reduced by a value of not less than 1.0 logs within the minimum recommended disinfection time with no increase at not less than four times the minimum recommended disinfection time within an experimental error of +/−0.5 logs. Secondary acceptance criteria for bacteria requires that there is a combined log reduction for the mean values of all three bacteria of not less than 5.0 logs within the recommended disinfection period. The minimum acceptable mean log reduction for any single bacterial type is 1.0 log. Stasis for the yeast and mold must be observed for the minimum recommended disinfection period. Results of the ISO/FDA microbial biocidal efficacy testing of the subject test solutions are set forth below in Table 2.

TABLE 2
Biocidal Efficacies With 10 Percent Organic Soil

| | | Log Reduction of | | | | |
|---|---|---|---|---|---|---|
| | | Sample | | | | |
| ISO Agent | Hours | 1 | 2 | 3 | 4 | 5 |
| Staphylococcus aureus | 1 | 1.4 | 1.9 | 3.0 | 4.3 | >4.7 |
| (ATCC 6538) | 2 | 1.6 | 2.6 | 3.8 | 4.1 | >4.7 |
| | 3 | 1.7 | 3.5 | >4.7 | >4.7 | >4.7 |
| | 4 | 2.3 | 3.6 | >4.7 | >4.7 | >4.7 |
| Pseudomonas aeruginosa | 1 | 2.5 | 3.7 | >4.7 | >4.7 | >4.7 |
| (ATCC 9027) | 2 | >4.7 | >4.7 | >4.7 | >4.7 | >4.7 |
| | 3 | >4.7 | >4.7 | >4.7 | >4.7 | >4.7 |
| | 4 | >4.7 | >4.7 | >4.7 | >4.7 | >4.7 |
| Serratia marcescens | 1 | 1.4 | 2.5 | 3.2 | 4.6 | >4.6 |
| (ATCC 13880) | 2 | 2.5 | 3.6 | 4.2 | >4.6 | >4.6 |
| | 3 | 3.1 | 4.5 | 4.5 | >4.6 | >4.6 |
| | 4 | 3.0 | 4.4 | >4.6 | >4.6 | >4.6 |
| Candida albicans | 1 | 0.9 | 1.3 | 2.1 | 3.1 | 3.6 |
| (ATCC 10231) | 2 | 1.4 | 2.3 | 3.3 | 4.4 | 4.3 |
| | 3 | 1.5 | 2.7 | 3.9 | 4.5 | 4.5 |
| | 4 | 1.5 | 3.3 | 4.5 | >4.5 | >4.5 |
| Fusarium solani | 1 | 3.8 | 3.8 | 3.8 | 4.1 | >4.1 |
| (ATCC 36031) | 2 | >4.1 | >4.1 | >4.1 | >4.1 | >4.1 |
| | 3 | >4.1 | >4.1 | >4.1 | >4.1 | >4.1 |
| | 4 | >4.1 | >4.1 | >4.1 | >4.1 | >4.1 |

### EXAMPLE 3

### Biocidal Testing of Test Samples With Five of FDA/ISO Challenge Microorganisms

Test solutions prepared in accordance with Example 1 above, were each tested for ISO/FDA microbial biocidal efficacy without organic soil using five FDA/ISO challenge microorganisms, i.e., three bacteria and two fungi. Primary acceptance criteria established for bacteria require that the number of viable bacteria, recovered per ml, shall be reduced by a value not less than 3.0 logs within the minimum recommended disinfection period. Primary acceptance criteria established for yeasts and molds require that the number of viable yeasts and molds, recovered per ml, shall be reduced by a value of not less than 1.0 logs within the minimum recommended disinfection time with no increase at not less than four times the minimum recommended disinfection time within an experimental error of +/0.5 logs. Secondary acceptance criteria for bacteria requires that there is a combined log reduction for the mean values of all three bacteria of not less than 5.0 logs within the recommended disinfection period. The minimum acceptable mean log reduction for any single bacterial type is 1.0 log. Stasis for the yeast and mold must be observed for the minimum recommended disinfection period. Results of the ISO/FDA microbial biocidal efficacy testing of the subject test solutions are set forth below in Table 3.

TABLE 3
Biocidal Efficacies Without Organic Soil

| | | Log Reduction of | | | | |
|---|---|---|---|---|---|---|
| | | Sample | | | | |
| ISO Agent | Hours | 1 | 2 | 3 | 4 | 5 |
| Staphylococcus aureus | 1 | 1.3 | 2.3 | 2.9 | 4.1 | >4.7 |
| (ATCC 6538) | 2 | 1.7 | 3.1 | 4.4 | >4.7 | >4.7 |
| | 3 | 2.3 | 4.1 | 4.6 | 4.7 | >4.7 |
| | 4 | 3.1 | 4.6 | 4.6 | >4.7 | >4.7 |
| Pseudomonas aeruginosa | 1 | 4.1 | >4.6 | >4.6 | >4.6 | >4.6 |
| (ATCC 9027) | 2 | >4.6 | >4.6 | >4.6 | >4.6 | >4.6 |
| | 3 | >4.6 | >4.6 | >4.6 | >4.6 | >4.6 |
| | 4 | >4.6 | >4.6 | >4.6 | >4.6 | >4.6 |
| Serratia marcescens | 1 | 1.4 | 2.3 | 3.2 | 4.5 | 4.6 |
| (ATCC 13880) | 2 | 2.3 | 4.1 | >4.7 | >4.7 | >4.7 |
| | 3 | 3.5 | >4.7 | >4.7 | >4.7 | >4.7 |
| | 4 | 4.7 | >4.7 | >4.7 | >4.7 | >4.7 |
| Candida albicans | 1 | 1.0 | 1.3 | 1.8 | 2.8 | 3.4 |
| (ATCC 10231) | 2 | 1.3 | 1.6 | 3.2 | 4.3 | >4.6 |
| | 3 | 1.3 | 1.8 | 3.6 | >4.6 | >4.6 |
| | 4 | 1.4 | 2.5 | 4.0 | 4.6 | >4.6 |
| Fusarium solani | 1 | 2.2 | 3.0 | 3.4 | >4.3 | 4.3 |
| (ATCC 36031) | 2 | 3.6 | >4.3 | >4.3 | >4.3 | >4.3 |
| | 3 | 4.2 | >4.3 | >4.3 | >4.3 | >4.3 |
| | 4 | >4.3 | >4.3 | >4.3 | >4.3 | >4.3 |

### EXAMPLE 4

### Biocidal Testing of Test Samples With Five of FDA/ISO Challenge Microorganisms

Test solutions prepared in accordance with Example 1 above, were each tested for ISO/FDA microbial biocidal efficacy with 10 percent organic soil using five FDA/ISO challenge microorganisms, i.e., three bacteria and two fungi. Primary acceptance criteria established for bacteria require that the number of viable bacteria, recovered per ml, shall be reduced by a value not less than 3.0 logs within the minimum recommended disinfection period. Primary acceptance criteria established for yeasts and molds require that the number of viable yeasts and molds, recovered per ml, shall be reduced by a value of not less than 1.0 logs within the minimum recommended disinfection time with no increase at not less than four times the minimum recommended disinfection time within an experimental error of +/−0.5 logs. Secondary acceptance criteria for bacteria requires that there is a combined log reduction for the mean values of all three bacteria of not less than 5.0 logs within the recommended disinfection period. The minimum acceptable mean log reduction for any single bacterial type is 1.0 log. Stasis for the yeast and mold must be observed for the minimum recommended disinfection period. Results of the ISO/FDA microbial biocidal efficacy testing of the subject test solutions are set forth below in Table 4.

TABLE 4
Biocidal Efficacies With 10 Percent Organic Soil

| | | Log Reduction of | | | | |
|---|---|---|---|---|---|---|
| | | Sample | | | | |
| ISO Agent | Hours | 6 | 7 | 8 | 9 | 10 |
| Staphylococcus aureus | 1 | 1.2 | 1.2 | 1.3 | 1.6 | 2.1 |
| (ATCC 6538) | 2 | 1.2 | 1.4 | 1.6 | 2.0 | 2.6 |
| | 3 | 1.2 | 1.5 | 1.7 | 2.6 | 2.9 |
| | 4 | 1.4 | 1.6 | 2.1 | 2.9 | 3.7 |
| Pseudomonas aeruginosa | 1 | 4.4 | >4.7 | >4.7 | >4.7 | >4.7 |
| (ATCC 9027) | 2 | >4.7 | >4.7 | >4.7 | >4.7 | >4.7 |
| | 3 | >4.7 | >4.7 | >4.7 | >4.7 | >4.7 |
| | 4 | >4.7 | >4.7 | >4.7 | >4.7 | >4.7 |
| Serratia marcescens | 1 | 1.0 | 1.2 | 1.5 | 1.7 | 1.8 |
| (ATCC 13880) | 2 | 1.3 | 1.7 | 2.4 | 2.6 | 2.8 |
| | 3 | 1.8 | 2.4 | 2.7 | 3.3 | 3.7 |
| | 4 | 2.4 | 2.6 | 3.3 | 4.0 | 4.6 |
| Candida albicans | 1 | 1.2 | 1.4 | 1.7 | 1.7 | 2.5 |
| (ATCC 10231) | 2 | 1.4 | 1.7 | 2.4 | 2.8 | 3.4 |
| | 3 | 1.6 | 2.1 | 2.8 | 3.4 | 3.8 |
| | 4 | 1.8 | 2.5 | 3.2 | 3.6 | 4.4 |
| Fusarium solani | 1 | 4.3 | 4.3 | 4.2 | >4.4 | >4.4 |
| (ATCC 36031) | 2 | >4.4 | >4.4 | >4.4 | >4.4 | >4.4 |
| | 3 | >4.4 | >4.4 | >4.4 | >4.4 | >4.4 |
| | 4 | >4.4 | >4.4 | >4.4 | >4.4 | >4.4 |

### EXAMPLE 5

### Biocidal Testing of Test Samples with Five of FDA/ISO Challenge Microorganisms

Test solutions prepared in accordance with Example 1 above, were each tested for ISO/FDA microbial biocidal efficacy with 10 percent organic soil using five FDA/ISO challenge microorganisms, i.e., three bacteria and two fungi. Primary acceptance criteria established for bacteria require that the number of viable bacteria, recovered per ml, shall be reduced by a value not less than 3.0 logs within the minimum recommended disinfection period. Primary acceptance criteria established for yeasts and molds require that the number of viable yeasts and molds, recovered per ml, shall be reduced by a value of not less than 1.0 logs within the minimum recommended disinfection time with no increase at not less than four times the minimum recommended disinfection time within an experimental error of +/−0.5 logs. Secondary acceptance criteria for bacteria requires that there is a combined log reduction for the mean values of all three bacteria of not less than 5.0 logs within the recommended disinfection period. The minimum acceptable mean log reduction for any single bacterial type is 1.0 log. Stasis for the yeast and mold must be observed for the minimum recommended disinfection period. Results of the ISO/FDA microbial biocidal efficacy testing of the subject test solutions are set forth below in Table 5.

TABLE 5
Biocidal Efficacies With 10 Percent Organic Soil

| | | Log Reduction of | | |
|---|---|---|---|---|
| | | Sample | | |
| ISO Agent | Hours | 11 | 12 | 13 |
| Staphylococcus aureus | 1 | 1.3 | 1.4 | 1.6 |
| (ATCC 6538) | 2 | 1.3 | 1.8 | 2.3 |
| | 3 | 1.5 | 2.3 | 2.7 |
| | 4 | 1.8 | 2.8 | 3.6 |
| Pseudomonas aeruginosa | 1 | 4.7 | 4.7 | 4.7 |
| (ATCC 9027) | 2 | >4.7 | >4.7 | >4.7 |
| | 3 | >4.7 | >4.7 | >4.7 |
| | 4 | >4.7 | >4.7 | >4.7 |
| Serratia marcescens | 1 | 1.3 | 1.4 | 1.6 |
| (ATCC 13880) | 2 | 1.6 | 1.9 | 2.7 |
| | 3 | 2.0 | 2.6 | 3.5 |
| | 4 | 2.5 | 3.5 | 3.8 |
| Candida albicans | 1 | 1.3 | 1.5 | 1.8 |
| (ATCC 10231) | 2 | 1.4 | 2.3 | 2.6 |
| | 3 | 1.7 | 2.4 | 3.4 |
| | 4 | 1.6 | 2.7 | 3.6 |
| Fusarium solani | 1 | 4.0 | 4.2 | 4.2 |
| (ATCC 36031) | 2 | >4.2 | >4.2 | >4.2 |
| | 3 | >4.2 | >4.2 | >4.2 |
| | 4 | >4.2 | >4.2 | >4.2 |

### EXAMPLE 6

### Biocidal Testing of Test Samples With Five of FDA/ISO Challenge Microorganisms

Test solutions prepared in accordance with Example 1 above, were each tested for ISO/FDA microbial biocidal efficacy with 10 percent organic soil using five FDA/ISO challenge microorganisms, i.e., three bacteria and two fungi. Primary acceptance criteria established for bacteria require that the number of viable bacteria, recovered per ml, shall be reduced by a value not less than 3.0 logs within the minimum recommended disinfection period. Primary acceptance criteria established for yeasts and molds require that the number of viable yeasts and molds, recovered per ml, shall be reduced by a value of not less than 1.0 logs within the minimum recommended disinfection time with no increase at not less than four times the minimum recommended disinfection time within an experimental error of +/−0.5 logs. Secondary acceptance criteria for bacteria requires that there is a combined log reduction for the mean values of all three bacteria of not less than 5.0 logs within the recommended disinfection period. The minimum acceptable mean log reduction for any single bacterial type is 1.0 log. Stasis for the yeast and mold must be observed for the minimum recommended disinfection period. Results of the ISO/FDA microbial biocidal efficacy testing of the subject test solutions are set forth below in Table 6.

TABLE 6
Biocidal Efficacies With 10 Percent Organic Soil

| | | Log Reduction of | | | |
|---|---|---|---|---|---|
| | | Sample | | | |
| ISO Agent | Hours | 14 | 15 | 16 | 17 |
| Staphylococcus aureus | 1 | 2.4 | 2.1 | 3.1 | 2.9 |
| (ATCC 6538) | 4 | 3.8 | 4.3 | >4.8 | 4.8 |
| Pseudomonas aeruginosa | 1 | >4.6 | >4.6 | >4.6 | >4.6 |
| (ATCC 9027) | 4 | >4.6 | >4.6 | >4.6 | >4.6 |
| Serratia marcescens | 1 | 1.9 | 1.9 | 2.7 | 3.3 |
| (ATCC 13880) | 4 | 4.6 | 4.4 | >4.6 | >4.6 |
| Candida albicans | 1 | 2.6 | 2.2 | 2.9 | 3.0 |
| (ATCC 10231) | 4 | 4.5 | 3.7 | 4.7 | 4.4 |
| Fusarium solani | 1 | 2.8 | 3.7 | >4.1 | >4.1 |
| (ATCC 36031) | 4 | >4.1 | 4.1 | >4.1 | >4.1 |

Cationic polysaccharide containing compositions of the present invention are useful as contact lens care solutions for disinfecting contact lenses. A disinfecting amount of antimicrobial agent is an amount that will at least partially reduce the microorganism population in the formulations employed. Preferably, a disinfecting amount is that which will reduce the microbial burden of representative bacteria by two log orders in four hours and more preferably by one log order in one hour. Most preferably, a disinfecting amount is an amount that will eliminate the microbial burden on a contact lens when used according to its regimen for the recommended soaking time (FDA Chemical Disinfection Efficacy Test—July 1985 Contact Lens Solution Draft Guidelines). Typically, such agents are present in concentrations ranging from about 0.00001 to about 0.5 percent weight/volume (w/v), and more preferably, from about 0.00003 to about 0.5 percent w/v. Unexpectedly, in the presence of one or more cationic polysaccharides a smaller amount of antimicrobial agent, i.e., a 5 to 30 percent reduction and more preferably a 15 to 30 percent reduction, is required to achieve a disinfecting amount.

As stated above, contact lenses are disinfected by contacting the lens with a solution of one or more compositions of the present composition. Although this may be accomplished by simply soaking a lens in the subject solution, greater cleaning can be achieved if a few drops of the solution are initially placed on each side of the lens, and rubbing the lens for a period of time, for example, approximately 20 seconds. The lens can then be subsequently immersed within several milliliters of the subject solution. Preferably, the lens is permitted to soak in the solution for at least four hours. The lenses are then removed from the solution, rinsed with the same or a different solution, for example a preserved isotonic saline solution and then replaced on the eye.

Solutions containing one or more compositions of the present invention may be formulated into specific contact lens care products for use as customary in the field of ophthalmology. Such products include but are not limited to wetting solutions, soaking solutions, cleaning and conditioning solutions, as well as multipurpose type lens care solutions and in-eye cleaning and conditioning solutions.

While the invention has been described in conjunction with specific examples thereof, this is illustrative only. Accordingly, many alternatives, modifications, and variations will be apparent to those skilled in the art in the light of the foregoing description and it is, therefore, intended to embrace all such alternatives, modifications, and variations as to fall within the spirit and scope of the appended claims.

1. Compositions Comprising:
one or more cationic polysaccharides and a disinfecting amount at a reduced amount of one or more antimicrobial agents.

2. The compositions of claim 1 wherein said one or more cationic polysaccharides are selected from the group consisting of variations of polyquaternium-10.

3. The compositions of claim 1 wherein said one or more cationic polysaccharides are selected from the group consisting of Polymer JR 125, Polymer JR 400, Polymer JR 30M, Polymer LR 400, Polymer LR 30 M and Polymer LK.

4. The compositions of claim 1 wherein said one or more antimicrobial agents are selected from the group consisting of 1,1′-hexamethylene-bis[5-(p-chlorophenyl)biguanide], water soluble salts of 1,1′-hexamethylene-bis[5-(p-chlorophenyl)biguanide], 1,1′-hexamethylene-bis[5-(2-ethylhexyl)biguanide], water soluble salts of 1,1′-hexamethylene-bis[5-(2-ethylhexyl)biguanide], poly(hexamethylene biguanide), water soluble salts of poly(hexamethylene biguanide), polyquaternium-1 and quaternary ammonium esters.

5. The composition of claim 1 wherein said one or more antimicrobial agents include 1,1′-hexamethylene-bis[5-(p-chlorophenyl)biguanide].

6. The composition of claim 1 wherein said disinfecting amount said one or more antimicrobial agents is a standard amount reduced by 5 to 30 percent.

7. A method of producing compositions of claim 1 comprising:
combining one or more cationic polysaccharides with a disinfecting amount at a reduced amount of one or more antimicrobial agents.

8. The method of claim 7 wherein said one or more cationic polysaccharides are selected from the group consisting of variations of polyquaternium-10.

9. The method of claim 7 wherein said one or more cationic polysaccharides are selected from the group consisting of Polymer JR 125, Polymer JR 400, Polymer JR 30M, Polymer LR 400, Polymer LR 30 M and Polymer LK.

10. The method of claim 7 wherein said one or more antimicrobial agents are selected from the group consisting of 1,1′-hexamethylene-bis[5-(p-chlorophenyl)biguanide], water soluble salts of 1,1′-hexamethylene-bis[5-(p-chlorophenyl)biguanide], 1,1′-hexamethylene-bis[5-(2-ethylhexyl)biguanide], water soluble salts of 1,1′-hexamethylene-bis[5-(2-ethylhexyl)biguanide], poly(hexamethylene biguanide), water soluble salts of poly(hexamethylene biguanide), polyquaternium-1 and quaternary ammonium esters.

11. The method of claim 7 wherein said one or more antimicrobial agents include 1,1′-hexamethylene-bis[5-(p-chlorophenyl)biguanide].

12. The method of claim 7 wherein said disinfecting amount said one or more antimicrobial agents is a standard amount reduced by 5 to 30 percent.

13. A solution comprising one or more compositions of claim 1.

14. The solution of claim 13 wherein said solution includes one or more buffers or buffering systems.

15. The solution of claim 13 wherein said solution includes one or more tonicity agents.

16. The solution of claim 13 wherein said solution includes one or more surfactants.

17. The solution of claim 13 wherein said solution includes one or more viscosity agents.

18. A method of using the solution of claim 13 comprising:
contacting a surface of a contact lens with said solution for a period of time suitable to eliminate a microbial burden on said contact lens.

19. A method of producing the solution of claim 13 comprising:
adding a reduced volume disinfecting amount of said one or more antimicrobial agents and one or more cationic polysaccharides to a solution.
